(19) United States
(12) Patent Application Publication
(10) Pub. No.: US 2023/0000744 A1
(43) Pub. Date: Jan. 5, 2023

(54) COMPOSITIONS AND METHODS FOR STYLING HAIR

(71) Applicant: L'Oreal, Paris (FR)

(72) Inventors: Rachel FEREBEE MAHER, Pittsburgh, PA (US); Allison PERNER, Metuchen, NJ (US); Vibha K. SHAH, Branchburg, NJ (US)

(21) Appl. No.: 17/853,580

(22) Filed: Jun. 29, 2022

Related U.S. Application Data
(60) Provisional application No. 63/217,183, filed on Jun. 30, 2021.

(30) Foreign Application Priority Data
Sep. 20, 2021 (FR) 2109872

(51) Int. Cl. A61K 8/37; A61Q 5/04; A61Q 5/06; A61K 8/73; A61K 8/34; A61K 8/67

(52) U.S. Cl. CPC A61K 8/375; A61Q 5/04; A61Q 5/06; A61K 8/73; A61K 8/345; A61K 8/67

(57) ABSTRACT

The disclosure relates to compositions comprising (a) at least one polyphenol; (b) at least one first gum chosen from carrageenans; (c) at least one second gum; (d) at least one humectant; (e) optionally, at least one buffer agent; and (f) at least one solvent. The compositions can be used for styling hair, for example temporarily straightening the hair and/or reducing frizz of the hair. The disclosure also relates to methods of using the compositions to style hair, straighten hair, and/or reduce frizz of hair.

### TECHNICAL FIELD

The present disclosure relates to compositions and methods for styling hair, such as curly or wavy hair. The compositions provide temporary straightening properties to the hair. The disclosure also relates to methods of using the compositions to style hair.

### BACKGROUND

Consumers with curly or wavy hair often desire to temporarily straighten their hair for a change in appearance. In addition, reducing the curls of very curly hair may increase the manageability and ease of styling of such hair.

Traditional hair-straightening products generally include heavy galenics or oils, which may leave an undesirable greasy or oily feeling. Products that provide hold, such as styling gels, can also give straightening effects, but these products typically flake on the hair. In addition, many hair straightening products involve heat or chemical treatments that may cause damage to the hair fibers and/or irritate the scalp.

As such, consumers desire new and improved compositions that can provide straightening effects to curly or wavy hair, and at the same time impart various additional advantageous properties to the hair such as softness, smoothness, shine, and/or frizz control.

It has now surprisingly been found that compositions comprising a particular combination of components as described herein are able to temporarily straighten curly or wavy hair without the use of heat or harsh chemicals, while at the same time delivering additional advantageous sensorial benefits to the hair.

### SUMMARY

The present disclosure relates to compositions for styling hair, such as curly or wavy hair. The compositions may provide beneficial effects such as straightening the hair and/or one or more of softness, smoothness, shine, and/or frizz control to the hair. The compositions are optionally leave-in compositions, and may, in various embodiments, be in the form of spray compositions.

The hair styling compositions may comprise, in certain embodiments: (a) at least one polyphenol; (b) at least one first gum chosen from carrageenans; (c) at least one second gum; (d) at least one humectant; (e) optionally, at least one buffer agent; and (f) at least one solvent.

In various embodiments, the at least one polyphenol is chosen from flavonoids and non-flavonoids, as well as mixtures thereof, and in a particular embodiment is chosen from flavonoids. For example, in various embodiments the at least one polyphenol comprises, consists essentially or, or consists of one or more flavonoid compounds. In at least one embodiment, the flavonoid is tannic acid. For example, in various embodiments the at least one polyphenol comprises, consists essentially or, or consists of tannic acid. The at least one polyphenol may be present in an amount ranging from about 0.01% to about 15%, from about 0.01% to about 10%, from about 3% to about 8%, from about 0.5% to about 6%, or from about 0.1% to about 2% by weight, relative to the total weight of the composition.

In certain embodiments, compositions according to the disclosure may comprise the at least one first gum in an amount ranging from about 0.01% to about 5%, such as from about 0.01% to about 2%, or from about 0.1% to about 0.5%, and the at least one second gum in an amount ranging from about 0.01% to about 3%, such as from about 0.01% to about 1%, or from about 0.4% to about 0.6%, all weights by weight based on the total weight of the composition. In certain embodiments, the first gum comprises, consists essentially of, or consists of carrageenan, and the second gum is chosen from acacia, agar, algin, alginic acid, ammonium alginate, amylopectin, calcium alginate, carnitine, dextrin, diutan gum, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, propylene glycol alginate, sclerotium gum, sodium carboxymethyl dextran, tragacanth gum, xanthan gum, biosaccharide gum, or mixtures thereof, such as, for example, sclerotium gum, xanthan gum, diutan gum, or mixtures thereof.

In various embodiments, the weight ratio of first gum:second gum ranges from about 1:1 to about 1:10, for example from about 1:1.2 to about 1:7 or about 1:1.3 to about 1:6.

In certain embodiments, the at least one humectant is chosen from glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof. Optionally, the at least one humectant comprises inulin and at least one glycol, wherein the weight ratio of inulin:glycol ranges from about 1:10 to about 1:25, or from about 1:10 to about 1:20.

The solvent comprises water and optionally at least one non-aqueous solvent, and may be present in an amount ranging from about 50% to about 99%, such as about 60% to about 98%, or about 70% to about 95% by weight, relative to the total weight of the composition. In at least one embodiment, the solvent consists of or consists essentially of water, with the solvent comprising less than about 20%, less than about 10%, less than about 5%, or less than about 3% by weight, relative to the total weight of the composition, of non-aqueous solvents.

In certain embodiments, the compositions comprise one or more buffer agents. Exemplary buffer agents that may be used include acids, such as citric, acetic, and maleic acid, and phosphate buffers such as monosodium phosphate and disodium phosphate. In one embodiment, the compositions comprise at least one acid and at least one phosphate buffer, such as, for example, citric acid and disodium phosphate. For example, the at least one buffer agent may include at least one acid present in an amount ranging from about 0.05% to about 3%, and at least one phosphate buffer present in an amount ranging from about 0.1% to about 5%, all weights based on the total weight of the composition.

In one embodiment, the hair styling composition comprises (a) tannic acid; (b) a first gum comprising carrageenan; (c) at least one second gum chosen from sclerotium gum, xanthan gum, diutan gum, or mixtures thereof; (d) at least one humectant chosen from glycerin, at least one glycol, inulin, sorbitol, or mixtures thereof; (e) a buffer agent chosen from citric acid, disodium phosphate, or mixtures thereof; and (f) water, present in an amount of at least 50% by weight, relative to the total weight of the composition, optionally wherein the weight ratio of carrageenan:second gum ranges from about 1:1 to about 1:8.

In another embodiment, the hair styling composition comprises (a) tannic acid, present in an amount ranging from about 0.5% to about 6%; (b) a first gum comprising carrageenan, present in an amount ranging from about 0.01% to about 1%; (c) about 0.2% to about 0.8% of at least one second gum chosen from sclerotium gum, xanthan gum, diutan gum, or mixtures thereof; (d) at least one glycol and inulin, wherein the weight ratio of inulin:glycol ranges from about 1:10 to about 1:20; (e) about 1% to about 5% of a buffer component chosen from citric acid, disodium phosphate, or mixtures thereof; and (f) water, present in an amount of at least 60%, wherein all weights are relative to the total weight of the hair styling composition; and optionally wherein the weight ratio of carrageenan:second gum ranges from about 1:1 to about 1:8.

The disclosure further relates to methods of using the compositions to style hair, such as methods of straightening hair, and/or methods of reducing frizz, wherein the methods comprise applying the composition to wet, damp, or dry hair.

For example, in various embodiments, the disclosure relates to methods of styling the hair, methods of straightening the hair, and/or methods of reducing frizz of the hair, where the methods comprise applying to the hair a composition comprising: (a) at least one polyphenol; (b) at least one first gum chosen from carrageenans; (c) at least one second gum; (d) at least one humectant; (e) optionally, at least one buffer agent; and (f) at least one solvent comprising water. In various embodiments of methods of styling, straightening, and/or reducing frizz of the hair, the at least one polyphenol may be chosen from flavonoid or non-flavonoid compounds, for example tannic acid, and may be present in an amount ranging from about 0.01% to about 15%, from about 0.01% to about 10%, from about 3% to about 8%, from about 0.5% to about 6%, or from about 0.1% to about 2% by weight, relative to the total weight of the composition. In various embodiments of methods of styling, straightening, and/or reducing frizz of the hair, the at least one first gum is carrageenan, and the second gum is chosen from acacia, agar, algin, alginic acid, ammonium alginate, amylopectin, calcium alginate, carnitine, dextrin, diutan gum, gelatin, gellan gum, guar gum, hectorite, hyaluronic acid, hydrated silica, hydroxypropyl chitosan, hydroxypropyl guar, karaya gum, kelp, locust bean gum, natto gum, potassium alginate, propylene glycol alginate, sclerotium gum, sodium carboxymethyl dextran, tragacanth gum, xanthan gum, biosaccharide gum, or mixtures thereof, such as, for example, from of sclerotium gum, xanthan gum, diutan gum, or mixtures thereof, wherein the at least one first gum is present in an amount ranging from about 0.01% to about 5%, such as from about 0.01% to about 2%, or from about 0.1% to about 0.5%, and the at least one second gum in an amount ranging from about 0.01% to about 3%, such as from about 0.01% to about 1%, or from about 0.4% to about 0.6%, all weights by weight based on the total weight of the composition. In at least certain embodiments of methods of styling, straightening, and/or reducing frizz of the hair, the weight ratio of first gum:second gum ranges from about 1:1 to about 1:10, for example from about 1:1.2 to about 1:7 or about 1:1.3 to about 1:6. The solvent in the methods of styling, straightening, and/or reducing frizz of the hair comprises water and optionally at least one non-aqueous solvent, and may be present in an amount ranging from about 50% to about 99%, such as about 60% to about 98%, or about 70% to about 95% by weight, relative to the total weight of the composition. In at least one embodiment, the solvent consists of or consists essentially of water, with the solvent comprising less than about 20%, less than about 10%, less than about 5%, or less than about 3% by weight, relative to the total weight of the composition, of non-aqueous solvents.

Optionally, in order to style, straighten, and/or reduce frizz of the hair, the compositions are left on the hair for a period of a few hours to a few days, for example until the hair is washed. In various embodiments, the compositions and/or methods do not include the use of a reducing agent, and/or do not include a step of heating the hair.

The compositions are not mascara compositions, and are not intended to be applied to the eyelashes or eyebrows. Similarly, the compositions are not make-up compositions, and are not intended to be applied to the skin.

### BRIEF DESCRIPTION OF THE FIGURE

The accompanying FIGURE, which is incorporated herein and constitute part of this specification, illustrate exemplary embodiments of the disclosure, and, together with the general description given above and the description provided herein, serve to demonstrate features of the disclosure.

FIG. 1 is a photograph showing a head of curly mannequin hair treated with compositions 1B (right half-head) and C1 (left half-head) before application of the compositions (T0), once the compositions have dried on the hair (T1), and after the hair is combed with the fingers to break the film formed by the compositions upon drying (T2).

It is to be understood that the foregoing and following descriptions are exemplary and explanatory only, and are not intended to be restrictive of any subject matter claimed.

### DETAILED DESCRIPTION

The disclosure relates to compositions for styling the hair, for example compositions for straightening wavy or curly hair and/or reducing frizz of the hair, as well as to methods of using the compositions.

### I. Compositions

In various embodiments, compositions according to the disclosure comprise (a) at least one polyphenol; (b) at least one first gum chosen from carrageenans; (c) at least one second gum; (d) at least one humectant; (e) optionally, at least one buffer compound; and (f) at least one solvent. The compositions are optionally leave-in compositions, and may, in various embodiments, be in the form of spray compositions.

Compositions according to the disclosure may provide straightening effects to the hair, e.g. temporary straightening effects, and in at least some embodiments may provide one or more additional benefits such as softness, smoothness, shine, and/or frizz control to the hair.

### Polyphenols

Compositions according to the disclosure comprise at least one polyphenol. Polyphenols are phenols with more than one phenolic —OH group that have the ability to act as “donor molecules” by donating their alcoholic hydrogen or accepting delocalized electrons. Useful polyphenols include both flavonoids and non-flavonoids.

Exemplary and non-limiting flavonoid compounds that can be used include, for example, flavanols, such as catechin, fisetin, kaempferol, myricetin, quercetin, rutin, proanthocyanidins, pyroanthocyanidins, theaflavins, or thearubigins (or thearubrins); dihydroflavonols, such as astilbin, dihydroquercetin, or silibinin; flavanones, such as hesperidin, neohesperidin, hesperetin, naringenin, naringin, or poncirin; flavones, such as apigenin, baicalin, diosmin, or rhoifolin; tannins, such as ellagitannins, tannic acid, gallic acid, or ellagic acid; isoflavonoids, such as biochanin A, Daidzein, or Genistein; fulvic acid, and neoflavonoids, as well as combinations thereof.

Exemplary and non-limiting non-flavonoid compounds that can be used include, for example, stibenoids such as astringin, resveratrol, or rhaponticin, as well as combinations thereof.

Other polyphenols that can be used include hydroxycinnamic acids, for example, chlorogenic acid, verbascoside; phenolic aldehydes; phenylpropenes; coumarins, coumestans, or tyrosols, as well as combinations thereof. In one embodiment, the polyphenols may be plant-based and/or organic.

In certain exemplary embodiments, polyphenols useful according to the disclosure may be chosen from tannic acid, resveratrol, catechin, ellagic acid, resorcinol, gallic acid, humic acid, chlorogenic acid, quercetin, anthocyanin, chebulinic acid, or mixtures thereof. In certain embodiments, the polyphenols are chosen from polyphenols that do not impart color to the hair. In at least one embodiment, the polyphenol comprises tannic acid.

In various embodiments, the at least one polyphenol is present in the compositions in an amount of about 0.01% to about 10% by weight, such as about 0.1% to about 8% or about 0.5% to about 5% by weight, relative to the total weight of the composition. By way of non-limiting example, the at least one polyphenol is present in the composition with a total amount of polyphenols ranging from about 0.1% to about 10%, from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.75% to about 10%, from about 0.75% to about 9%, from about 0.75% to about 8%, from about 0.75% to about 7%, from about 0.75% to about 6%, from about 0.75% to about 5%, from about 0.75% to about 4%, or from about 0.75% to about 3% by weight, relative to the total weight of the composition, including all ranges and subranges thereof.

In other embodiments, the at least one polyphenol is present in an amount ranging from about 0.01% to about 2.5%, from about 0.01% to about 2.4%, from about 0.01% to about 2.3%, from about 0.01% to about 2.2%, from about 0.01% to about 2.1%, from about 0.01% to about 2%, from about 0.01% to about 1.9%, from about 0.01% to about 1.8%, from about 0.01% to about 1.7%, from about 0.01% to about 1.6%, from about 0.01% to about 1.5%, from about 0.01% to about 1.4%, from about 0.01% to about 1.3%, from about 0.01% to about 1.2%, from about 0.01% to about 1.1%, from about 0.01% to about 1%, from about 0.1% to about 2.5%, from about 0.1% to about 2.4%, from about 0.1% to about 2.3%, from about 0.1% to about 2.2%, from about 0.1% to about 2.1%, from about 0.1% to about 2%, from about 0.1% to about 1.9%, from about 0.1% to about 1.8%, from about 0.1% to about 1.7%, from about 0.1% to about 1.6%, from about 0.1% to about 1.5%, from about 0.1% to about 1.4%, from about 0.1% to about 1.3%, from about 0.1% to about 1.2%, from about 0.1% to about 1.1%, from about 0.1% to about 1%, from about 0.5% to about 2.5%, from about 0.5% to about 2.4%, from about 0.5% to about 2.3%, from about 0.5% to about 2.2%, from about 0.5% to about 2.1%, from about 0.5% to about 2%, from about 0.5% to about 1.9%, from about 0.5% to about 1.8%, from about 0.5% to about 1.7%, from about 0.5% to about 1.6%, from about 0.5% to about 1.5%, from about 0.5% to about 1.4%, from about 0.5% to about 1.3%, from about 0.5% to about 1.2%, from about 0.5% to about 1.1%, or from about 0.5% to about 1% by weight, relative to the total weight of the composition, including all ranges and subranges thereof.

In some embodiments, the at least one polyphenol comprises, consists essentially of, or consists of tannic acid, where the total amount of tannic acid in the composition ranges from about 0.1% to about 10%, such as 0.1% to about 7%, about 0.5%% to about 7%, or about 0.75% to about 6% by weight, relative to the total weight of the composition, including all ranges and subranges thereof.

In further embodiments, the at least one polyphenol comprises, consists essentially of, or consists of tannic acid, where the total amount of tannic acid in the composition ranges from about 0.1% to about 5%, such as 0.1% to about 3%, about 0.5%% to about 2%, or about 0.75% to about 1.5% by weight, relative to the total weight of the composition, including all ranges and subranges thereof.

### Gums

Compositions according to the disclosure comprise at least one first gum chosen from carrageenans, and at least one second gum.

Non-limiting examples of carrageenans that may be chosen for the first gum include carrageenan, calcium carrageenan, potassium carrageenan, sodium carrageenan, and mixtures thereof.

The first gum may be present in an amount ranging from about 0.001% to about 10%, such as from about 0.01% to about 5%, about 0.05% to about 3%, about 0.1% to about 2%, or about 0.1% to about 0.5%, based on the total weight of the composition. For example, the first gum may range from about 0.01% to about 5%, about 0.01% to about 4%, about 0.01% to about 3%, about 0.01% to about 2%, about 0.01% to about 1%, about 0.01% to about 0.5%, about 0.05% to about 5%, about 0.05% to about 4%, about 0.05% to about 3%, about 0.05% to about 2%, about 0.05% to about 1%, about 0.05% to about 0.5%, about 0.1% to about 5%, about 0.1% to about 4%, about 0.1% to about 3%, about 0.1% to about 2%, about 0.1% to about 1%, about 0.1% to about 0.5%, or about 0.1% to about 0.3% by weight, including all ranges and sub-ranges there between, based on the total weight of the composition. In certain embodiments, the first gum may be present in an amount of ranging from about 0.1% to about 1.5%, such as about 0.1% to about 1.4%, about 0.1% to about 1.3%, about 0.1% to about 1.2%, about 0.1% to about 1.1%, about 0.1% to about 1%, about 0.1% to about 0.9%, about 0.1% to about 0.8%, about 0.1% to about 0.7%, about 0.1% to about 0.6%, about 0.1% to about 0.5%, about 0.1% to about 0.4%, or about 0.1% to about 0.3% by weight, including all ranges and sub-ranges there between, based on the total weight of the composition.

Non-limiting examples of gums that may be chosen for the at least one second gum include acacia, agar, algin, alginic acid, ammonium alginate, amylopectin, calcium alginate, carboxymethyl hydroxyethylcellulose, carnitine, cellulose acetate propionate carboxylate, cellulose gum, dextrin, diutan gum, gelatin, gellan gum, guar gum, hydroxyethylcellulose, hydroxypropylcellulose, hydroxypropyl methylcellulose, hydroxypropyl chitosan, hydroxypropyl guar, karaya gum, kelp, locust bean gum, methyl hydroxyethylcellulose, microcrystalline cellulose, natto gum, pectin, potassium alginate, propylene glycol alginate, sclerotium gum, sodium carboxymethyl dextran, sodium cellulose sulfate, tragacanth gum, xanthan gum, biosaccharide gum, or mixtures thereof. In certain embodiments, the at least one second gum comprises, consists essentially of, or consists of sclerotium gum, xanthan gum, diutan gum, or mixtures thereof.

The at least one second gum may be present in an amount ranging from about 0.01% to about 3%, such as from about 0.1% to about 2%, about 0.2% to about 1%, about 0.3% to about 0.8%, or about 0.3% to about 0.7%, based on the total weight of the composition. For example, the at least one second gum may range from about 0.01% to about 5%, about 0.01% to about 4%, about 0.01% to about 3%, about 0.01% to about 2%, about 0.01% to about 1%, about 0.01% to about 0.5%, about 0.05% to about 5%, about 0.05% to about 4%, about 0.05% to about 3%, about 0.05% to about 2%, about 0.05% to about 1%, about 0.05% to about 0.5%, about 0.1% to about 5%, about 0.1% to about 4%, about 0.1% to about 3%, about 0.1% to about 2%, about 0.1% to about 1%, about 0.1% to about 0.5%, or about 0.1% to about 0.3% by weight, including all ranges and sub-ranges there between, based on the total weight of the composition. In certain embodiments, the at least one second gum may be present in an amount ranging from about 0.1% to about 1%, such as about 0.1% to about 0.8%, about 0.1% to about 0.7%, about 0.1% to about 0.6%, about 0.1% to about 0.5%, about 0.1% to about 0.4%, or about 0.1% to about 0.3% by weight, including all ranges and sub-ranges there between, based on the total weight of the composition. In one embodiment, the at least one second gum is present in an amount ranging from about 0.2% to about 0.8%, about 0.3% to about 0.7%, or about 0.4% to about 0.6% by weight, based on the total weight of the composition. In various embodiments, the at least one second gum comprises, consists essentially of, or consists of sclerotium gum, xanthan gum, diutan gum, or mixtures thereof, and is present in an amount ranging from about 0.1% to about 1%, such as about 0.4% to about 0.6% by weight, based on the total weight of the composition.

In certain embodiments, it may be desirable to choose the amount of first gum and at least one second gum to be present in the compositions in a particular ratio, for optimum styling and beneficial properties. For example, the ratio of first gum:second gum may range from about 1:1 to about 1:10, such as about 1:1.1 to about 1:9, about 1:1.15 to about 1:8, about 1:1.2 to about 1:7, about 1:1.25 to about 1:6.5, or about 1:1.3 to about 1:6, including all ranges and subranges thereof. In one embodiment, the ratio of first gum:second gum ranges from about 1:1 to about 1:8.

In some embodiments, the compositions comprise at least one first gum comprising, consisting essentially of, or consisting of carrageenan, and at least one second gum comprising, consisting essentially of, or consisting of sclerotium gum, xanthan gum, diutan gum, or mixtures thereof, wherein the amounts of first gum and second gum are chosen so that the ratio of first gum:second gum ranges from about 1:1.2 to about 1:75, or from about 1:1.3 to about 1:6. In one embodiment, the ratio of carrageenan:second gum ranges from about 1:1 to about 1:8.

### Humectants

Compositions according to the disclosure comprise at least one humectant. In certain embodiments, compositions according to the disclosure comprise at least two humectants.

Non-limiting humectant compounds that can be chosen include, for example, polyhydric alcohols, such as C₂-C₈ or C₃-C₆ polyhydric alcohols. By way of example, glycerol (glycerin), propylene glycol, butylene glycol, pentylene glycol, hexylene glycol, dipropylene glycols, diethylene glycol, diglycerol, or mixtures thereof, may be chosen.

Additional non-limiting examples of humectants that can be chosen include hyaluronic acid, sugars such as sorbitol or xylitol, or beta-fructans, for example inulin. Mixtures of two or more humectants can also be chosen.

The humectants can be present in the composition in a total amount ranging from about 0.1% to about 20%, such as from about 0.5% to about 20%, about 1% to about 18%, or about 5% to about 15% by weight, based on the total weight of the composition, including all ranges and subranges thereof. Individually, the humectants may be present in an amount ranging from about 0.1% to about 20%, such as about 0.3% to about 17%, or about 0.5% to about 15% by weight, based on the total weight of the composition, including all ranges and subranges thereof.

In one exemplary embodiment, the composition comprises one or more C₂-C₈ polyhydric alcohols, present in a total amount ranging from about 1% to about 20%, such as from about 5% to about 18%, or about 8% to about 15% by weight, based on the total weight of the composition, including all ranges and subranges thereof. In yet a further embodiment, the composition comprises one or more sugars, present in a total amount ranging from about 1% to about 10%, such as about 2% to about 8%, or about 3% to about 7% by weight, based on the total weight of the composition, including all ranges and subranges thereof. In a still further embodiment, the composition comprises at least one beta-fructan, present in an amount ranging from about 0.1% to about 2%, such as about 0.1% to about 1.5%, or about 0.1% to about 1% by weight, based on the total weight of the composition, including all ranges and subranges thereof.

In various exemplary embodiments, the composition comprises glycerin in an amount ranging from about 1% to about 6%, such as about 2% to about 5%, or about 3% to about 4% by weight, based on the total weight of the composition, including all ranges and subranges thereof, including all ranges and subranges thereof; at least one glycol, for example butylene glycol, in an amount ranging from about 1% to about 15%, such as about 3% to about 12%, about 5% to about 12%, or about 5% to about 10% by weight, based on the total weight of the composition, including all ranges and subranges thereof; inulin in an amount ranging from about 0.1% to about 2%, such as about 0.1% to about 1%, or about 0.25% to about 0.75% by weight, based on the total weight of the composition, including all ranges and subranges thereof; and/or sorbitol, in an amount ranging from about 1% to about 10%, such as about 2% to about 8%, or about 4% to about 6% by weight, based on the total weight of the composition, including all ranges and subranges thereof.

In one exemplary and non-limiting embodiment, the composition comprises inulin and at least one glycol, wherein the ratio of inulin:glycol ranges from about 1:10 to about 1:25, such as about 1:10 to about 1:22, about 1:10 to about 1:20, about 1:15 to about 1:25, about 1:15 to about 1:22, or about 1:15 to about 1:20.

### Buffer

Optionally, compositions according to the disclosure comprise at least one buffer compound or agent. In various embodiments, compositions according to the disclosure comprise at least two buffer compounds or agents.

In various exemplary embodiments, the buffer agent(s) may be chosen from acids, for example citric acid, maleic acid, or acetic acid, bases such as sodium and calcium hydroxide, and phosphate buffers such as monosodium phosphate or disodium phosphate.

If present, the buffer agents may be present in a total amount ranging from about 0.1% to about 10%, such as about 0.5% to about 8%, about 1% to about 5%, or about 2% to about 4% by weight, based on the total weight of the composition, including all ranges and subranges thereof.

In an exemplary and non-limiting embodiment, the compositions comprise at least one acid buffer, such as citric acid, present in an amount ranging from about 0.05% to about 3%, such as about 0.1% to about 2%, or about 0.5% to about 1.5% by weight, based on the total weight of the composition, including all ranges and subranges thereof; and/or at least one phosphate buffer, such as disodium phosphate, present in an amount ranging from about 0.1% to about 5%, such as about 0.5% to about 4%, or about 1% to about 3% by weight, based on the total weight of the composition, including all ranges and subranges thereof.

### Solvent

Compositions according to the disclosure comprise one or more cosmetically acceptable solvents. The solvent(s) may be chosen from water, non-aqueous solvents, or mixtures thereof. The total amount of solvent in the composition may range from about 50% to about 99% by weight, relative to the total weight of the composition, including all ranges and subranges therebetween. For example, in one embodiment, the total amount of solvent may be about 50% to about 98%, about 60% to about 95%, about 70% to about 90%, about 70% to about 85%, or about 75% to about 85% by weight, relative to the total weight of the composition.

In one embodiment, the solvent comprises water. In other embodiments, the composition comprises water and one or more non-aqueous solvents. For example, C₁-C₄ alcohols, organic solvents, polyols, glycols, hydrocarbons/alkanes, glycol ethers, or any mixture thereof. Non-limiting examples of solvents which may be used include alkane polyols such as, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol (isopropyl alcohol); glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-isopropyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-isopropyl ether, dipropylene glycol monomethyl ether, dipropylene glycol monoethyl ether, dipropylene glycol mono-n-propyl ether, and dipropylene glycol mono-isopropyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In some exemplary embodiments, the solvent comprises water and at least one additional solvent chosen from linear or branched, saturated or unsaturated, and substituted or unsubstituted polyols, for example C₂-C₈ polyols or C₃-C₆ polyols, such as diols and/or triols. Any stereoisomer of the polyols may be used. In various exemplary embodiments, the C₂-C₈ polyols may be chosen from glycols such as ethylene glycol, propylene glycol, butylene glycol, hexylene glycol, pentylene glycol, 1,3-propanediol, diethylene glycol, triethylene glycol, tetraethylene glycol, pentaethylene glycol, dipropylene glycol, caprylyl glycol, glycerin, diglycerin, and mixtures thereof.

In various embodiments, the solvent consists of, or consists essentially of, water. In certain embodiments, the solvent comprises water in an amount ranging from about 50% to about 95%, about 60% to about 90%, about 70% to about 85%, or about 75% to about 80%, by weight of the solvent, with the remainder of the solvent comprising one or more additional solvents. For example, in certain embodiments, the composition comprises about 50% to about 99%, about 60% to about 98%, about 70% to about 95%, or about 75% to about 93% water, by weight of the composition. In other embodiments, the compositions comprise water and at least one non-aqueous solvent, where the total amount of non-aqueous solvent is present in an amount less than about 20%, such as less than about 18%, less than about 16%, less than about 15%, less than about 13%, less than about 12%, less than about 10%, less than 8%, less than 5%, or less than 3% by weight, relative to the total weight of the composition. In some embodiments, the compositions are free or substantially free of non-aqueous solvents.

### Additional Components

Compositions according to the disclosure may optionally comprise any additional or auxiliary component suitable for use in such compositions. Such components may include, but are not limited to, conditioning agents, moisturizing agents, fillers, structuring agents, shine agents, antioxidants, penetrants, sequestrants, fragrances, dispersants, plant extracts, preserving agents (preservatives), opacifiers, sunscreen agents, pH adjusting agents, vitamins, and antistatic agents.

Optional additional or auxiliary components may be present in an amount ranging up to about 15%, such as from about 0.01% to about 15%, about 0.1% to about 10%, or about 0.1% to about 5% by weight, relative to the total weight of the composition.

In various embodiments, the compositions may have a pH less than or equal to about 7, such as from about 3 to about 6.8, from about 3.5 to about 6.5, from about 4 to about 6.5, from about 4.5 to about 6.5, from about 5 to about 6.5 or from about 5.5 to about 6.5. In other embodiments, the pH of the compositions may be less than or equal to about 6, such as from about 2 to about 6, from about 2 to about 5, or from about 3 to about 5. In some embodiments, the pH of the compositions may be substantially unchanged (e.g. may have a change in pH of less than 20%, less than 15%, or less than 10%) when stored under ambient conditions for 5 days or more, such as 7 days or more, or 9 days or more.

In various embodiments, the compositions may have a viscosity ranging from about 140 mPa·s to about 400 mPa·s, such as about 160 mPa·s to about 375 mPa·s, when measured using a Rheomat RM 180, M2 spindle for 30 seconds under ambient conditions. In some embodiments, the viscosity of the compositions may be substantially unchanged (e.g. may have a change in viscosity of less than 20%, less than 15%, or less than 10%) when stored under ambient conditions for 3 days or more, such as 7 days or more, or 10 days or more.

Compositions according to the disclosure are typically in the form of a liquid, for example a liquid that is suitable for application as a spray-on composition, or may be a sprayable gel. The composition may be an aerosol, in which case a propellant may be used, or may be in non-aerosol form, such as a pump-type spray.

### II. Methods

The present disclosure also relates to methods for styling hair, for example by temporarily straightening curly or wavy hair by application of the compositions described herein to the hair. The methods generally comprise applying any of the compositions according to the disclosure to the hair.

The compositions may be useful in a variety of settings, either for chemically treated or untreated hair, and/or for natural or unnatural curls. The degree of curliness or curl type of the hair may vary and is not limited. For example, the curls of hair been treated by the compositions disclosed herein may range from slightly wavy to very kinky and coily hair, and may have different textures and colors.

In addition to temporarily straightening the hair, the compositions are surprisingly capable of providing a variety of desirable sensory benefits, for example, moisture, smoothness, softness, shine, and/or anti-frizz properties, to the hair. As such, the compositions are useful in styling (which includes straightening) hair while also caring for hair, conditioning hair, and/or imparting one or more above-described sensory benefits to the hair.

Typically, methods according to the disclosure may include applying an effective amount of a composition disclosed herein to hair having waves or curls, when the hair is dry, wet, damp, or moist. The hair may be either natural or synthetic hair. As used herein, the term “effective amount” refers to an amount sufficient to provide a desired straightening effect to the hair, taking into account the degree of curliness, the length, the volume, and the texture of the hair, and/or an amount sufficient to provide a desired sensory benefit to the hair. In general, from about 0.5 grams to about 50 grams of product is applied to the hair, depending on the specific product formulation, hair length, hair volume, and hair style type. The composition applied to the hair may, for example, be distributed evenly by combing through with fingers or a means such as a comb or the like. The composition may then be dried, for example air-dried or dried with a hair dryer. Heat may optionally be used during drying, but is not required. The composition may be allowed to remain on the hair as a leave-in product for any period of time as needed, for example, a few hours or a few days, or until the next washing or rinsing of the hair. Alternatively, the compositions may be rinsed from the hair after a brief leave-in period, from about a few seconds (e.g. 5, 10, 20, 30, or 60 seconds) to about 10, 20, or 30 minutes, or longer.

In some embodiments, before applying the composition to the hair, the hair may be first cleansed with a commercially available shampoo, and/or rinsed with water, and/or dried, either partially or completely. The composition may then be applied to the washed and/or rinsed hair when the hair is wet, damp, moist, or dry. In some other embodiments, before applying the composition to dry hair, the hair can optionally be moistened, damped, or wetted by water spray or using a wet towel, or by applying other treatment compositions that make the hair moist, damp, or wet.

It has surprisingly and unexpectedly been discovered that methods according to the disclosure generally do not require the use of a reducing agent, including a base, or heating the hair in order to achieve a temporary straightening effect. As such, in some embodiments, the method does not include using a reducing agent or a base, and/or does not including heating the hair.

In various embodiments, the methods of treating hair with the compositions according to the disclosure impart the benefits described above to the hair, without a greasy feel or flaking, relative to hair not having been treated with a composition according to the disclosure. The benefits imparted to the hair may remain a desired length of time, such as a few hours, a few days, or until the hair is rinsed or washed.

The compositions described herein are not mascara compositions, i.e. are not intended to be applied to eyelashes and/or eyebrows. The compositions are likewise not makeup compositions, i.e. are not intended to be applied to skin, in particular, to skin on the face, body, or lips. Thus, treatment of, or application to, the hair is intended to be limited to hair on the head, i.e. the scalp of the head, and not eyelashes, eyebrows, body or facial hair, or the like. In such embodiments, a skilled person understands that “hair on the head” means hair that grows from the scalp of the head, and excludes eyebrows, eyelashes, facial hair, etc.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense. The phrase “consisting essentially of” limits the scope of a claim to the specified materials or steps and those that do not materially affect the basic and novel characteristics of the compositions.

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof. Likewise, the term “a salt thereof” also relates to “salts thereof.” Thus, where the disclosure refers to “an element selected from the group consisting of A, B, C, D, E, F, a salt thereof, or mixtures thereof,” it indicates that that one or more of A, B, C, D, or F may be included, one or more of a salt of A, a salt of B, a salt of C, a salt of D, a salt of E, or a salt of F may be included, or a mixture of any two of A, B, C, D, E, F, a salt of A, a salt of B, a salt of C, a salt of D, a salt of E, and a salt of F may be included. However, it is to be understood that such language also includes embodiments where G, H, I, etc., are present, as long as at least one of A, B, C, D, E, F, or a salt thereof, is also present.

The term “and/or” should be understood to include both the conjunctive and the disjunctive. For example, “A and/or B” means “A and B” as well as “A or B,” and expressly covers instances of either, without reference to the other. For example, a composition that comprises “at least one acid buffer and/or at least one phosphate buffer” means that the disclosure includes compositions comprising one or more acid buffers or one or more phosphate buffers, as well as compositions comprising one or more acid buffers and one or more phosphate buffers. As a further example, disclosure of “methods of styling the hair, straightening the hair, and/or reducing frizz of the hair” includes methods of styling the hair or methods of straightening the hair or methods of reducing frizz of the hair, as well as methods of styling the hair and straightening the hair, methods of styling the hair and reducing frizz of the hair, methods of styling the hair and straightening the hair and reducing frizz of the hair, etc.

As used herein, the phrases “and mixtures thereof,” “and a mixture thereof,” “and combinations thereof,” “and a combination thereof,” “or mixtures thereof,” “or a mixture thereof,” “or combinations thereof,” “or a combination thereof,” and variations thereof are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not so expressly stated. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

Further, unless expressly stated otherwise or when the context of the disclosure clearly dictates otherwise, the language “the at least one X may be present in an amount ranging from Y % to Z %” is to be understood to mean that the total amount of X's present in the composition ranges from Y % to Z %, inclusive of the endpoints. For example, “the at least one polyphenol may be present in an amount ranging from about 0.01% to about 10%” means that the total amount of polyphenols present ranges from about 0.01% to about 10%, inclusive of both 0.01% and 10%.

“Active material” as used herein with respect to the percent amount of an ingredient or raw material, refers to 100% activity of the ingredient or raw material.

All amounts given herein are relative to the amount of active material, unless otherwise indicated.

Unless otherwise indicated, all percentages herein are by weight, relative to the total weight of the composition.

All percentages, parts and ratios herein are based upon the total weight of the compositions of the present disclosure, unless otherwise indicated.

As used herein, the term “treat” (and its grammatical variations, such as “treating”) refers to the application of the compositions of the present disclosure onto the surface of the hair on the head.

As used herein, the phrase “applying a composition onto hair” and variations thereof is intended to mean contacting the hair with at least one of the compositions of the disclosure, in any manner. It may also mean contacting the hair in an effective amount.

As used herein, the term “styling” is intended to include “shaping” and “straightening.”

As used herein, the term “curly hair” refers to any hair including a curl, and “wavy hair” refers to any hair including a wave. The curl and/or wave may be natural or unnatural, i.e., formed by chemical treatment or physical treatment of the hair. The degree of curliness and/or waviness of the hair may vary and is not limited.

A “leave-in” composition or product refers to a hair-treatment composition that is not rinsed and/or washed away with water or acceptable solvent after the application of the composition onto the hair; instead, the composition is allowed to remain on the hair for a period of time as desired, such from 1 hour, 2 hours, 3 hours, 4 hours, up to 8 hours, overnight, or as long as needed, until next time of washing or rinsing the hair.

As used herein, the term “polyol” refers to an organic molecule comprising at least two free hydroxyl groups.

The terms “substantially without” or “essentially without” as used herein means the specific material may be used in a manufacturing process in small amounts that do not materially affect the basic and novel characteristics of the compositions according to the disclosure. The terms may also mean that the specific material is not used in a manufacturing process but may still be present in a raw material that is included in the composition.

As used herein, unless expressly stated otherwise, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the term “salts” refers to throughout the disclosure may include salts having a counter-ion, in either ionized or un-ionized form. It is to be understood that, with regard to salts of acids described herein, it is intended to encompass the use of a salt of the acid as an ingredient added to a composition according to the disclosure, or to the salt of the acid that forms when the acid is used as an ingredient in a composition according to the disclosure (in ionized or un-ionized form).

As used herein, the term “synthetic” means a material that is not of natural origin. The term “natural” and “naturally-sourced” means a material of natural origin, such as derived from plants, which also cannot be subsequently chemically or physically modified. “Plant-based” means that the material came from a plant.

As used herein, the term “temporary” should be understood to indicate that some degree of benefit or effect is imparted to the hair that remains from when the hair is treated with a composition according to the disclosure, until the composition is removed from the hair, e.g. by washing the hair.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

### EXAMPLES

The following examples are intended to be non-limiting and explanatory in nature only. In the Examples, amounts are expressed in percentage by weight (wt %) of active materials, relative to the total weight of the composition, unless otherwise noted.

### Example 1—Compositions

The inventive and comparative compositions set forth in Table 1 below were prepared.

TABLE 1
Compositions

| | Inventive | | | Comparative |
|---|---|---|---|---|
| | 1A | 1B | 1C | C1 |
| BUTYLENE GLYCOL | 10 | 10 | 10 | |
| TANNIC ACID | 5 | 0.9 | 0.9 | 5 |
| SCLEROTIUM GUM | 0.5 | 0.5 | 0.5 | |
| GLYCERIN | 3.75 | 3.75 | 3.75 | |
| CITRIC ACID | | | 1.29 | |
| HYDROGENATED STARCH | 3.5 | 3.5 | 3.5 | |
| HYDROLYSATE | | | | |
| DISODIUM PHOSPHATE | | | 2.76 | |
| CARRAGEENAN | 0.5 | 0.3 | 0.3 | 0.5 |
| INULIN | 0.49 | 0.49 | 0.49 | |
| PRESERVATIVES | 0.7 | 0.7 | 0.7 | 0.7 |
| SOLVENTS (water + | QS to 100 | QS to 100 | QS to 100 | QS to 100 |
| non-aqueous solvents) | | | | |

The compositions were prepared as follows. First, the solvent phase was heated to a temperature of about 70-75° C. The gum and polyphenol components were added individually with mixing at medium speed using a rotor-stator blade on a Rayneri V2004 mixer. Once the polyphenol and gums were fully dispersed, the rest of the ingredients were added one by one with mixing at medium speed to ensure each was fully incorporated, and an emulsion was formed. Then the batch was cooled to a temperature of about 30-35° C., while mixing with a propeller-type blade.

Each of compositions 1A-1C and C1 provided temporary straightening benefits to hair, and reduced frizz even in high humidity. However, hair treated with composition C1 felt more stiff and coated compared to hair treated with compositions 1A-1C, which had a smoother, more natural feel.

### Example 2—Stability Testing

The stability of the inventive and comparative compositions in Table 1 was evaluated by measuring the pH, viscosity, and visual appearance of the compositions, over time.

TABLE 2A
pH

| | Day 0 | Day 5 | | | Day 9 | | |
|---|---|---|---|---|---|---|---|
| Composition | pH | T | pH | Δ pH | T | pH | Δ pH |
| C1 | 3.65 | 25° C. | 3.54 | −0.11 | 25° C. | 3.41 | −0.24 |
| | | 60° C. | 2.71 | −0.94 | 60° C. | 2.40 | −1.25 |
| 1A | 3.83 | 25° C. | 3.78 | −0.05 | 25° C. | 3.69 | −0.14 |
| | | 60° C. | 3.06 | −0.77 | 60° C. | 2.60 | −1.23 |
| 1B | 4.70 | 25° C. | 4.37 | −0.33 | 25° C. | 4.29 | −0.41 |
| | | 60° C. | 3.65 | −1.05 | 60° C. | 3.33 | −1.37 |
| 1C | 4.57 | 25° C. | 4.50 | −0.07 | 25° C. | 4.46 | −0.11 |
| | | 60° C. | 4.47 | −0.10 | 60° C. | 4.44 | −0.13 |

TABLE 2B
Viscosity

| | Day 0 | Day 3 | | | Day 10 | | |
|---|---|---|---|---|---|---|---|
| Composition | η (mPa · s) | T | η (mPa · s) | Δ η | T | η (mPa · s) | Δ η |
| C1 | Water thin | — | Water thin | — | — | Water thin | — |
| 1A | 287 | 25° C. | 254 | −33 | 25° C. | 256 | −31 |
| | | 60° C. | 99 | −188 | 60° C. | 99 | −188 |
| 1B | 283 | 25° C. | 245 | −38 | 25° C. | 228 | −55 |
| | | 60° C. | 122 | −161 | 60° C. | 110 | −173 |
| 1C | 375 | 25° C. | 385 | +10 | 25° C. | 315 | −60 |
| | | 60° C. | 375 | — | 60° C. | 282 | −93 |

The viscosity of each of compositions 1A-1C and C1 was measured using a Rheomat RM 180, M2 spindle for 30 seconds under ambient conditions (i.e. room temperature and pressure). Composition C1 had a viscosity upon preparation that was very low (water thin), which did not change over time. Each of inventive compositions 1A-1C had an initial viscosity (day 0) that was suitable for application to the hair, and a change in viscosity over time that was acceptable to demonstrate that the compositions were stable, with composition 1C demonstrating the greatest stability.

In addition, each of compositions 1A-1C and C1 was evaluated visually over a 10-day period. Comparative composition C1 showed that some solids precipitated out at day 5, which were visible at the bottom of the container, demonstrating that composition C1 has the least stability. Inventive compositions 1A and 1B had small amounts of solids suspended in the solutions at day 9, but no precipitate was noticeable on the bottom of the container. Inventive composition 1C was the most stable, with no solid precipitate visible either in the solution or at the bottom of the container still by day 10.

### Example 3—Testing on Hair

Compositions 1B and C1 were tested on a mannequin head with curly hair. First, the hair was washed and conditioned with a standard shampoo and conditioner, and then excess water was removed with a towel. Next, either composition 1B or C1 was applied to one-half of the mannequin head in an amount of 7.5 grams per half-head, and distributed through the hair by hand, after which the swatches were dried with a rollerball dryer at a temperature of about 40-45° C. for a period of about 60 minutes.

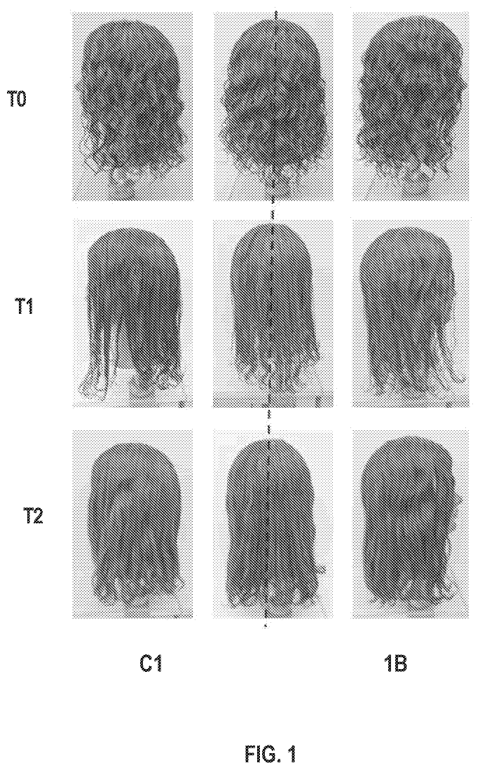

Once the hair was dry, the hair was combed with the fingers to break the film that had formed on the hair after drying. The hair treated with both compositions 1B and C1 demonstrated effective straightening of the hair, as seen in FIG. 1. In FIG. 1, TO shows the treated hair after the compositions have been applied, while still wet, T1 shows the treated hair after the compositions have dried on the hair, and T2 shows the treated hair after it is combed with the fingers to break the film formed upon drying.

It was noted that the straightening effect of composition 1B, with a significantly lower concentration of tannic acid, was as effective as that of composition C1, which is believed to be due to the additional components in composition 1B. Further, the hair treated with composition 1B had improved sensorial properties compared to the hair treated with composition C1, such as slip and glide, as well as smoothness and frizz-control of the treated hair.

The above Examples demonstrate that compositions according to the disclosure provide effective temporary straightening benefits to curly hair, while at the same time providing sensorial benefits such as smoothness and frizz-control to the hair. Further, compositions according to the disclosure have improved stability compared to compositions not according to the disclosure.

It will be apparent to those skilled in the art that various modifications and variations can be made in the compositions and methods according to the disclosure without departing from the spirit or scope of the disclosure. Thus, it is intended that the disclosure cover such modifications and variations and their equivalents.

1-27. (canceled)

28. A hair styling composition comprising:
(a) at least one polyphenol;
(b) at least one first gum chosen from carrageenans;
(c) at least one second gum;
(d) at least one humectant;
(e) optionally, at least one buffer agent; and
(f) at least one solvent comprising water.

29. The hair styling composition of claim 28, wherein the total amount of polyphenols ranges from about 0.01% to about 10% by weight, relative to the total weight of the composition.

30. The hair styling composition of claim 28, wherein the at least one polyphenol is chosen from flavonoid compounds.

31. The hair styling composition of claim 28, wherein the at least one polyphenol is tannic acid.

32. The hair styling composition of claim 28, wherein the total amounts of the at least one first gum and/or at least one second gum independently range from about 0.01% to about 5% by weight, relative to the total weight of the composition.

33. The hair styling composition of claim 28, comprising:
(b) carrageenan; and
(c) at least one second gum chosen from acacia, agar, algin, alginic acid, ammonium alginate, amylopectin, calcium alginate, carboxymethyl hydroxyethylcellulose, carnitine, cellulose acetate propionate carboxylate, cellulose gum, dextrin, diutan gum, gelatin, gellan gum, guar gum, hydroxyethylcellulose, hydroxypropylcellulose, hydroxypropyl methylcellulose, hydroxypropyl chitosan, hydroxypropyl guar, karaya gum, kelp, locust bean gum, methyl hydroxyethylcellulose, microcrystalline cellulose, natto gum, pectin, potassium alginate, propylene glycol alginate, sclerotium gum, sodium carboxymethyl dextran, sodium cellulose sulfate, tragacanth gum, xanthan gum, biosaccharide gum, or mixtures thereof.

34. The hair styling composition of claim 28, wherein the weight ratio of first gum:second gum ranges from about 1:1 to about 1:10.

35. The hair styling composition of claim 28, wherein the at least one humectant is chosen from glycerin, propylene glycol, butylene glycol, pentylene glycol, hexylene glycol, dipropylene glycol, diethylene glycol, diglycerol, hyaluronic acid, sorbitol, xylitol, inulin, or mixtures thereof.

36. The hair styling composition of claim 28, wherein the humectant comprises inulin and at least one glycol, and the weight ratio of inulin:glycol ranges from about 1:10 to about 1:25.

37. The hair styling composition of claim 28, comprising at least one buffer agent chosen from acids, phosphate buffers, or mixtures thereof.

38. The hair styling composition of claim 37, wherein the at least one buffer agent comprises:
at least one acid, present in an amount ranging from about 0.05% to about 3% by weight; and/or
at least one phosphate buffer, present in an amount ranging from about 0.1% to about 5% by weight,
wherein all amounts are by weight, relative to the total weight of the composition.

39. The hair styling composition claim 28, wherein the solvent comprises water in an amount of at least about 50% by weight, relative to the total weight of the composition.

40. The hair styling composition of claim 28, wherein the solvent comprises water and at least one non-aqueous solvent, wherein the total amount of non-aqueous solvent is less than about 20% by weight, relative to the total weight of the composition.

41. The hair styling composition of claim 28, wherein the composition is not a mascara composition or a makeup composition.

42. The hair styling composition of claim 28, further comprising at least one antioxidant, at least one preservative, at least one pH adjuster, at least one buffer, at least one vitamin, or a mixture of two or more thereof.

43. A hair styling composition for treating keratin fibers, comprising:
(a) tannic acid;
(b) a first gum comprising carrageenan;
(c) at least one second gum chosen from sclerotium gum, xanthan gum, diutan gum, or mixtures thereof;
(d) at least one humectant chosen from glycerin, at least one glycol, inulin, sorbitol, or mixtures thereof;
(e) a buffer agent chosen from citric acid, disodium phosphate, or mixtures thereof; and
(f) water, present in an amount of at least about 50% by weight, relative to the total weight of the composition.

44. The hair styling composition of claim 43, comprising:
(a) tannic acid, present in an amount ranging from about 0.5% to about 6%;
(b) a first gum comprising carrageenan, present in an amount ranging from about 0.01% to about 1%;
(c) about 0.2% to about 0.8% of at least one second gum chosen from sclerotium gum, xanthan gum, diutan gum, or mixtures thereof;
(d) at least one glycol and inulin, wherein the weight ratio of inulin:glycol ranges from about 1:10 to about 1:20;
(e) about 1% to about 5% of a buffer component chosen from citric acid, disodium phosphate, or mixtures thereof; and
(f) water, present in an amount of at least about 60%,
wherein all amounts are by weight, relative to the total weight of the composition.

45. The hair styling composition of claim 43, wherein the weight ratio of carrageenan:second gum ranges from about 1:1 to about 1:8.

46. A method for styling hair comprising applying to the hair a composition comprising:
(a) at least one polyphenol;
(b) at least one first gum chosen from carrageenans;
(c) at least one second gum;
(d) at least one humectant;
(e) optionally, at least one buffer agent; and
(f) at least one solvent comprising water.

47. The method for styling hair according to claim 46, wherein the composition applied to the hair does not comprise a reducing agent, and no step of heating the hair occurs after the composition is applied to the hair.
